(19) United States
(12) Patent Application Publication
(10) Pub. No.: US 2008/0008772 A1
(43) Pub. Date: Jan. 10, 2008

(54) Narcotic biphasic release compositions and methods for treatment of coughing, sneezing, rhinorrhea, and/or nasal obstruction

(75) Inventors: John A. Giordano, West Orange, NJ (US); Kevin M. Brown, Harrington Park, NJ (US)

Correspondence Address: Kirkpatrick & Lockhart Preston Gates Ellis LLP;Attn: Ellen Klann, 1601 K St. N.W., WASHINGTON, DC, 20006, US

(73) Assignee: Everett Laboratories, Inc.

(21) Appl. No.: 11/480,434

(22) Filed: Jul. 5, 2006

(51) Int. Cl. A61K 31/485; A61K 31/205; A01N 31/14; A61K 31/137; A61K 31/075

(52) U.S. Cl. 424/725.1; 514/282; 514/717; 514/554; 514/649

(57) ABSTRACT

The present invention relates to compositions that comprise immediate release and extended release guaifenesin, and extended release hydrocodone bitartrate. The present invention also includes methods for using these compositions for treatment of patients suffering from, for example and without limitation, coughing, sneezing, rhinorrhea, and/or nasal obstruction.

### FIELD OF THE INVENTION

The present invention relates to compositions comprising an immediate release expectorant, a controlled release expectorant, and a controlled extended release narcotic antitussive. Specifically, the compositions may comprise guaifenesin, and hydrocodone bitartrate. The present invention also includes methods for using these compositions for treatment of patients suffering from, for example and without limitation, coughing, sneezing, rhinorrhea, and/or nasal obstruction.

### BACKGROUND OF THE INVENTION

People suffering from coughing, sneezing, rhinorrhea, and/or nasal obstruction commonly take throat lozenges, cough syrups or cough drops for symptomatic relief. While many such medications presently exist, there is room for improvement in the composition of these medications. Many medications contain a combination or variety of antitussives, expectorants and/or decongestants. While such a combination or variety may be acceptable to some patients, others may have restrictions due to allergies or other incompatibilities with certain ingredients. Therefore, there is a need for a coughing, sneezing, rhinorrhea, and/or nasal obstruction medication that are restricted to the inclusion of specific antitussives and expectorants, and specifically with variable release profiles.

Guaifenesin is an expectorant that increases respiratory tract fluid secretions and helps to loosen phlegm and bronchial secretions. By reducing the viscosity of secretions, guaifenesin increases the efficiency of the mucocilary mechanism in removing accumulated secretions from the upper and lower airway. Guaifenesin promotes lower respiratory tract drainage by thinning bronchial secretions, lubricates irritated respiratory tract membranes through increased mucus flow, and facilitates removal of viscous, inspissated mucus. As a result, sinus and bronchial drainage is improved, and dry, nonproductive coughs become more productive and less frequent. Guaifenesin is readily absorbed from the gastrointestinal tract and is rapidly metabolized and excreted in the urine. Guaifenesin has a plasma half life of one hour. The major urinary metabolite is b-(2-methoxyphenoxy)lactic acid. Chemically, guaifenesin is 3-(2-methoxyphenoxy)-1,2 propanediol.

Hydrocodone bitartrate is an opioid analgesic and antitussive which occurs as fine, white crystals or as a crystalline powder. It is affected by light. It is soluble in water, slightly soluble in alcohol and insoluble in ether and in chloroform. The chemical name is: 4,5a-epoxy-3-methoxy-17-methylmorphinan-6-one tartrate (1:1)hydrate (2:5). Clinical trials have proven hydrocodone bitartrate to be an effective antitussive agent which is pharmacologically 2 to 8 times as potent as codeine. At equi-effective doses, its sedative action is greater than codeine. The precise mechanism of action of hydrocodone and other opiates is not known; however, hydrocodone is believed to act by directly depressing the cough center. The effects of hydrocodone in therapeutic doses on the cardiovascular system is insignificant. The constipation effects of hydrocodone are much weaker than that of morphine and no stronger than that of codeine. At therapeutic antitussive doses, it does exert analgesic effects.

### SUMMARY OF THE INVENTION

The present invention provides compositions and methods of using these compositions for the therapeutic treatment of coughing, sneezing, rhinorrhea, and/or nasal obstruction. Specifically, for example, the present invention relates to novel compositions of antitussives and expectorants that can be used to treat coughing, sneezing, rhinorrhea, and/or nasal obstruction caused by a variety of factors.

One aspect of the invention is a composition comprising hydrocodone bitartrate and/or guaifenesin which can be administered to a patient. In some embodiments, the composition is substantially free of other added active ingredients, such as another antitussive, such as codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol. In other embodiments, the composition is substantially free of another decongestant, in particular a nasal decongestant such as ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, phenylephrine and xylometazoline hydrochloride. In other embodiments, the composition is substantially free of another opioid analgesic, such as codeine, morphine, hydromorphone, oxymorphone, levorphanol, fentanyl, propoxyphene, diphenoxylate, meperidine, methadone, and oxycodone. In other embodiments, the composition is substantially free of another expectorant, such as ammonium chloride, ammonium carbonate, acetylcysteine, antimony potassium tartrate, glycerin, potassium iodide, sodium citrate, terpin hydrate, and tolu balsam.

In some embodiments, the composition is in a solid dosage form, specifically a caplet.

In some embodiments, guaifenesin comprises immediate release and/or controlled release forms. The controlled release form of guaifenesin may be guaiacolsulfonate and/or guaifenesin tannate. In some embodiments, the controlled release and immediate release forms of guaifenesin are formulated in an immediate release matrix. In other embodiments, the controlled release form of guaifenesin is formulated in an immediate release matrix and the dosage form may be coated with an immediate release coating containing guaifenesin.

In some embodiments, the hydrocodone bitartrate is in a controlled release form, such as hydrocodone polistirex or a resinate of hydrocodone, such as when complexed with sodium polystyrene sulfonate. The controlled release form of hydrocodone bitartrate may be formulated in an immediate release matrix.

In some embodiments, the dosage form is formulated so that hydrocodone bitartrate is released in a controlled release manner and guaifenesin is released in an immediate release and a controlled release manner. In some embodiments, substantially all of the drug(s) is released from the dosage form by about 12 hours after ingestion by a patient.

In some embodiments, the hydrocodone bitartrate is formulated in an immediate release matrix and the resulting dosage form is coated with a delay release coating, optionally with a more exterior coating of guaifenesin in an immediate release coating. In other embodiments, part or all of the guaifenesin is formulated in an immediate release matrix and the resulting dosage form is coated with a delay release coating, optionally with a more exterior coating of guaifenesin in an immediate release coating.

In other embodiments, the hydrocodone bitartrate and/or the guaifenesin is formulated into a particle comprising hydrocodone bitartrate and/or guaifenesin in an immediate release matrix with a delay release coating. In specific embodiments, the hydrocodone bitartrate and/or the guaifenesin particles are enclosed in a capsule or compressed into a tablet. The delay release coating may be of varied compositions and thicknesses, and may optionally have a more exterior coating guaifenesin in an immediate release coating. In more specific embodiments, the capsules or tablets also contain particles of guaifenesin in an immediate release matrix without a delay release coating.

In other embodiments, guaifenesin is formulated in an extended release matrix, and the dosage form may be optionally coated with guaifenesin in a immediate release matrix or coating. In other embodiments, hydrocodone bitartrate is formulated in an extended release matrix.

In other embodiment, the composition comprises about 2.7 mg per dose to about 8.3 mg per dose, about 4.1 mg per dose to about 6.9 mg per dose, about 5.0 mg per dose to about 6.1 mg per dose or about 5.2 mg per dose to about 5.8 mg per dose controlled release hydrocodone bitartrate. In other embodiments, the composition comprises about 200 mg per dose to about 600 mg per dose, about 350 mg per dose to about 450 mg per dose, about 360 mg per dose to about 440 mg per dose or about 380 mg per dose to about 420 mg per dose controlled release guaifenesin. In other embodiments, the composition comprises about 100 mg per dose to about 300 mg per dose, about 150 mg per dose to about 250 mg per dose, about 180 mg per dose to about 220 mg per dose, or about 190 mg per dose to about 210 mg per dose of immediate release guaifenesin. In a specific embodiment, the composition comprises about 5.2 mg per dose to about 5.8 mg per dose controlled release hydrocodone bitartrate; about 380 mg per dose to about 420 mg per dose controlled release guaifenesin; and about 190 mg per dose to about 210 mg per dose immediate release guaifenesin. In some embodiments, the composition may comprise about 5.5 mg per dose controlled release hydrocodone bitartrate, about 400 mg per dose controlled release guaifenesin and/or about 200 mg per dose immediate release guaifenesin.

Another aspect of the invention are methods comprising administering to a patient a composition comprising hydrocodone bitartrate and guaifenesin. In some embodiments, the composition is administered to the patient orally, specifically at a frequency of once a day, twice a day, three times a day or four times a day. In other specific embodiments, the composition is administered to the patient in a dose of about 0.5 to about three or more dosage forms. In other embodiments, the patient is suffering from one or more conditions selected from the group consisting of coughing, sneezing, rhinorrhea, nasal obstruction, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, a respiratory disease, a cold, acute bronchitis, chronic bronchitis, asthmatic bronchitis, bronchiectasis, pneumonia, lung tuberculosis, silicosis, silicotuberculosis, pulmonary cancer, upper respiratory inflammation, pharyngitis, laryngitis, nasal catarrh, asthma, bronchial asthma, infantile asthma, pulmonary emphysema, pneumoconiosis, pulmonary fibrosis, pulmonary silicosis, pulmonary suppuration, pleuritis, tonsillitis, cough hives, and whooping cough.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

### DEFINITIONS

It is understood that the present invention is not limited to the particular methodologies, protocols, fillers, excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a decongestant” is a reference to one or more decongestants and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “patient,” as used herein, comprises any and all organisms and includes the term “subject.” “Patient” may refer to a human or any other animal, including mammals.

The term “effective amount” means an amount of a compound/composition according to the present invention effective in producing the desired therapeutic effect.

The term “administrable” defines a composition that is able to be given to a patient. Likewise, “administering” refers to the act of giving a composition to a patient or otherwise making such composition available to a patient.

The term “active ingredient” as used herein is any ingredient that is an antitussive, a decongestant or an expectorant when taken orally by a patient.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

For example, “pharmaceutically acceptable salts” refer to derivatives of the disclosed compounds wherein the specified compound is converted to an acid or base salt thereof. Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic,. glutamic, benzoic, salicylic, sulfanilic, 2-acetoxybenzoic, fumaric, toluensulfonic, methanesulfonic, ethane dislfonic, oxalic, isethionic, and the like.

For purposes of the present invention the term “controlled release” or “modified release” refers to part of all of a dosage form that can release one or more active pharmaceutical agents in a profile that may extend over a period of time after administration (i.e., typically over a period of more than 1 hour from administration). Characteristic release profiles of controlled release (CR) may also be referred to as sustained release (SR), prolonged release (PR), extended release (ER or EX), and delayed release (DR). When used in association with the dissolution profiles discussed herein, the term “controlled release” may, in one embodiment, refer to that portion of a dosage form according to the present invention that delivers active agent over a period of time typically greater than 1 hour.

“Immediate release” refers to part or all of a dosage form that releases active agent substantially immediately upon contact with gastric juices and that results in substantially complete dissolution within about 1 hour. The characteristic of immediate release (IR) may also be referred to as instant release (IR). When used in association with the dissolution profiles discussed herein, the term “immediate release” refers to that portion of a dosage form according to the present invention that delivers active agent over a period of time less than 1 hour.

Initial peak plasma level refers to the first rise in blood plasma level of the active agent and may be followed by one or more additional peaks, one of which may be referred to as C_{MAX}. “C” is shorthand for concentration, “T” for time, “max” for maximum, and “min” for minimum. The term “C_{MAX}” is the peak blood plasma concentration exhibited by the compositions of the present invention. “T_{MAX}” refers to the time that C_{MAX} occurs in the plasma concentration-time profile. “C_{MIN}” is the minimum plasma concentration and “T_{MIN}” is the time that C_{MIN} occurs. Initial peak plasma level refers to the first rise in blood plasma level of the active agent and may be followed by one or more additional peaks, one of which may be C_{MAX}. As used herein, “mean maximum GABA_{B} agonist release” refers to the mean GABA_{B} agonist C_{MAX}.

The USP paddle method refers to the Paddle and Basket Method as described in United States Pharmacopoeia, Edition XXII (1990). In particular, the USP paddle method of 50 rpm or 75 rpm in 900 ml SGF or SIF at pH 1.2 or pH 6.8 at 37° C. may be used to determine the in vitro dissolution profiles according to the present invention.

“Mean plasma concentration-time profile” is the mathematical average of plasma concentration at each time point over a 24-hr period obtained in at least 12 healthy adult male and female subjects. Sampling times are 0.5, 1, 1.5, 2, 2.5, 3, 3.5, 4, 4.5, 5, 6, 7, 8, 10, 12, 16, and 24 hours.

The term “dose” or “dosage,” as used herein, is the amount of medication to be taken at one time.

The term “dose-equivalent amount,” as used herein, refers to an amount of a variant or pharmaceutically acceptable salt of an active compound that is an equivalent amount of the dose of the active compound per weight.

The term “dose unit,” as used herein, is the amount of the dosage form that is intended to be consumed to deliver the predetermined dosage to an adult human. For example, a dosage unit may be 1 or 2 caplets.

The term “dosage form,” as used herein, is the form in which the dose is to be administered to the patient. The drug is generally administered as part of a formulation that includes nonmedical agents, referred to as pharmaceutic ingredients. The dosage form has a unique physical and pharmaceutical characteristics. Dosage forms may be solid, liquid or gaseous. Solid forms include, but are not limited to tablets, caplets, lozenges, wafers etc. Liquid dosage forms include, but are not limited to syrups, elixirs, injectable solutions, and intravenous solutions. Gaseous forms include vapors, inhalants, and the like.

The term “excipients” refer to pharmacologically inert ingredients that are not active in the body. See HANDBOOK OF PHARMACEUTICAL EXCIPIENTS (Am. Pharm. Ass'n 1986). The artisan of ordinary skill in the art will recognize that many different excipients can be used in formulations according to the present invention and the list provided herein is not exhaustive.

The term “substantially free,” as used herein, means free from therapeutically effective amounts of compounds when administered in suggested dosages, but may include trace amounts of compounds in non-therapeutically effective amounts.

The term “substantially envelop” is intended to define the total or near-total enclosure of a component. Such an enclosure includes, preferably, at least about 80% enclosure, more preferably at least about 90% enclosure, and even more preferably at least about 99% enclosure.

The term “released in the stomach” means released at a pH consistent with the pH in a patients stomach. The rate and amount of release in the stomach may be ascertained in vitro using standard USP dissolution test or in vivo using actual patient studies.

The term “released in the intestine” means at a pH consistent with the pH in a patient's small intestine. The rate and amount of release in the intestine may be ascertained in vitro using standard USP dissolution test or in vivo using actual patient studies.

“After administration” refers to the time after the patient or study subject has taken, by oral administration, the drug-containing formulation.

“In vitro” refers to testing done outside of a patient's body, for example in special laboratory apparatus. For example, standard USP dissolution tests are known in the art and taught, for example, by the United States Pharmacopoeia, Edition XXII (1990). These include, for example, testing drug-containing formulations at 50 rpm or 75 rpm in 900 ml SGF or SIF at pH 1.2 or pH 6.8 at 37° C.

“In vivo” refers to testing performed in a subject's or patient's body.

“Steady state” refers to the repeated dosing of a drug until it reaches a stable level of absorption and elimination such that the amount of drug in the body is constant.

### COMPOSITIONS OF THE INVENTION

Through the inclusion of guaifenesin for immediate release, an extended-release guaifenesin and an extended-release hydrocodone bitartrate, the compositions and methods of the present invention may alleviate symptoms, such as coughing, sneezing, rhinorrhea, and/or nasal obstruction caused by a variety of factors.

In one embodiment, the composition of the invention may comprise guaifenesin, an extended-release form of guaifenesin, and an extended release form of hydrocodone bitartrate. In some embodiments, the extended release aspect of the extended release form may be due the presence of the guaifenesin and/or hydrocodone bitartrate in a variant form that retains the therapeutic properties of drug but has altered bioavailability of the drug in the digestive tract of the patient. In other embodiments, the extended release aspect of the extended release form may be due to formulation of guaifenesin and/or hydrocodone bitartrate with other compounds that alter the bioavailability of the drug in the digestive tract of the patient. Finally, in some embodiments, the extended release aspect may be due to a combination of variant form of the drug and the formulation of the drug.

In order to facilitate expectoration, medicines referred to as “expectorants” have been used. Most expectorants serve to remove the secretion by diluting it through an increase in secretion by the mucosa of the airway, promotion of separation from the mucosa and enhancement of ciliary beat. Guaifenesin (3-(2-methoxypphenoxy)-1,2-propanediol), also known as glyceryl guaiacolate, is an expectorant. It is readily absorbed from the intestinal tract and is thought to enter airway secretions unmetabolized and to have a direct effect either on the mucus secretion itself or the epithelium. Rubin, 116 CHEST 195-200 (1999). For example, guaifenesin is thought to reduce the thickness of mucus and phlegm secretions by increasing the production of fluids in the respiratory tract thus helping to liquefy and thin airway secretions. The increased flow of less viscid secretions promotes ciliary action and further facilitates the removal of airway secretions. Guaifenesin also may inhibit cough peripherally in the airway, by hydrating airway mucus so that it shields the cough receptors from cough-inducing irritants. Dicpinigaitis & Gayle, 124 CHEST 2178-2181 (2003). These peripheral actions of guaifenesin aid in the removal of accumulated secretions from the trachea, bronchi and lungs, thus changing a dry, non-productive cough to a cough that is more productive and less frequent. Guaifenesin also may act to suppress cough through an effect in the central nervous system. Rubin, supra. While the exact mechanism of this action of guaifenesin is not known, it is believed that guaifenesin acts centrally by depressing or blocking nerve impulse transmission at the internuncial neuron level of the subcortical areas of the brain, brainstem and spinal cord thus relaxing both the laryngeal and pharyngeal muscles.

In some embodiments, an extended-release form of guaifenesin may be used. One such extended release variant form of guaifenesin is potassium guaiacolsulfonate. Another such extended release form of guaifenesin is guaifenesin tannate. Methods to prepare and use guaifenesin tannate are provided in U.S. Pat. Nos. 6,689,817 and 6,677,381, both of which are incorporated by reference herein. It is contemplated that one or more of these compounds can be used to effect the extended-release aspect of the guaifenesin when used in the compositions of the invention in dose-equivalent amounts.

In a specific embodiment of the compositions and methods of the present invention, an immediate release form of guaifenesin may be included in amounts ranging from about 100 mg per dose to about 300 mg per dose. In another specific embodiment of the compositions and methods of the present invention, an immediate release form of guaifenesin may be included in amounts ranging from about 190 mg per dose to about 210 mg per dose. In another specific embodiment of the compositions and methods of the present invention, an immediate release form of guaifenesin may be included in amounts ranging from about 180 mg per dose to about 220 mg per dose. In another specific embodiment of the compositions and methods of the present invention, an immediate release form of guaifenesin may be included in amounts ranging from about 150 mg per dose to about 250 mg per dose. In another specific embodiment of the compositions and methods of the present invention, an immediate release form of guaifenesin may be included in an amount of about 200 mg per dose.

In a specific embodiment of the compositions and methods of the present invention, an extended-release form of guaifenesin may be included in amounts ranging from about 200 mg per dose to about 600 mg per dose. In another specific embodiment of the compositions and methods of the present invention, an extended-release form of guaifenesin may be included in amounts ranging from about 380 mg per dose to about 420 mg per dose. In another specific embodiment of the compositions and methods of the present invention, an extended-release form of guaifenesin may be included in amounts ranging from about 360 mg per dose to about 440 mg per dose. In another specific embodiment of the compositions and methods of the present invention, an extended-release form of guaifenesin may be included in amounts ranging from about 350 mg per dose to about 450 mg per dose. In another specific embodiment of the compositions and methods of the present invention, an extended-release form of guaifenesin may be included in an amount of about 400 mg per dose.

Antitussive drugs may act peripherally to inhibit cough by suppressing the responsiveness of one or more vagal sensory receptors that produce cough. Bolser et al., 86(3) J. APPL. PHYSIOL. 1017-1024 (1999); Adcock, RESPIR. MED. 85, Suppl. A 43-46 (1991); Bolser, 9 PULM. PHARMACOL. 357-364 (1997); Kase, 1 TRENDS PHARMACOL. SCI. 237-239 (1980). Antitussive drugs also may act within the central nervous system at the level of the brain stem, where the basic neural circuitry responsible for cough is located. Bolser et al., supra; Korpas & Tomori, COUGH AND OTHER RESPIRATORY REFLEXES, New York, Karger (1979); Shannon, 9 PULM. PHARMACOL. 343-347 (1997); Shannon et al., NEURAL CONTROL OF BREATHING, edited by Miller et al. 215-224 (1996). Specifically, centrally-acting antitussives are thought to inhibit cough by interfering with the central modulation of afferent signals from the periphery, thereby decreasing sensitivity of the cough center located within the medulla to incoming stimuli. Even more specifically, a recent model of the basic cough circuitry suggests that the eupneic respiratory pattern and the cough motor pattern are produced by essentially the same neural components. Although this pattern generator normally controls breathing, its behavior is modified to produce cough by excitatory inputs from medullary second-order interneurons mediating pulmonary rapidly and slowly adapting receptor (RAR and SAR, respectively)-afferent information. Shannon, supra; Shannon et al., supra. Centrally active antitussive drugs may act at any level within this system. For example, these drugs could suppress the responsiveness of components of the central pathway for transmitting vagal sensory information (second-order interneurons) and/or have more complex effects on the motor pattern generator for cough. Bolser & DeGennaro, 662 BRAIN RES. 25-30 (1994); Bolser et al., 113 BR. J. PHARMACOL. 1344-1348 (1994); Chou & Wang, 223 J. PHARMACOL. EXP. THER. 249-253 (1975).

Hydrocodone bitartrate is an opioid analgesic and antitussive which occurs as fine, white crystals or as a crystalline powder. It is affected by light. It is soluble in water, slightly soluble in alcohol and insoluble in ether and in chloroform. The chemical name is: 4,5a-epoxy-3-methoxy-17-methylmorphinan-6-one tartrate (1:1)hydrate (2:5). Clinical trials have proven hydrocodone bitartrate to be an effective antitussive agent, which is pharmacologically 2 to 8 times as potent as codeine. At equi-effective doses, its sedative action is greater than codeine. The precise mechanism of action of hydrocodone and other opiates is not known; however, hydrocodone is believed to act by directly depressing the cough center. The effects of hydrocodone in therapeutic doses on the cardiovascular system is insignificant. The constipation effects of hydrocodone are much weaker than that of morphine and no stronger than that of codeine. At therapeutic antitussive doses, it does exert analgesic effects.

In one specific embodiment of the compositions and methods of the present invention, a controlled-release form of hydrocodone bitartrate may be included in amounts ranging from about 2.7 mg per dose to about 8.3 mg per dose. In another specific embodiment of the compositions and methods of the present invention, a controlled-release form of hydrocodone bitartrate may be included in amounts ranging from about 5.2 mg per dose to about 5.8 mg per dose. In another specific embodiment of the compositions and methods of the present invention, a controlled-release form of hydrocodone bitartrate may be included in amounts ranging from about 5.0 mg per dose to about 6.1 mg per dose. In another specific embodiment of the compositions and methods of the present invention, a controlled-release form of hydrocodone bitartrate may be included in amounts ranging from about 4.1 mg per dose to about 6.9 mg per dose. In another specific embodiment of the compositions and methods of the present invention, a controlled-release form of hydrocodone bitartrate may be included in an amount of about 5.5 mg per dose.

In one embodiment, an extended release variant of hydrocodone bitartrate, such as hydrocodone polistirex may be used. Hydrocodone polistirex is a sulfonated styrene-divinyl benzene copolymer complex with 4,5α-epoxy-3-methoxy-17-methylmorphinan-6-one. In another embodiment, the extended release form is a drug resinate of hydrocodone. A “drug resinate” is a complex formed between a drug and an ion exchange resin. The complexation mechanism is salt formation. In a specific embodiment, the extended release variant form may be hydrocodone complexed with sodium polystyrene sulfonate (Amberlite™ IRP69, Rohm and Haas, Philadelphia, Pa.).

In some embodiments of the present invention, the compositions may be substantially free of active ingredients other than guaifenesin, and hydrocodone bitartrate. For example, in one embodiment, the compositions of the present invention may be substantially free of at least one other added antitussive. In another embodiment of the present invention, the compositions may be substantially free of at least one other added decongestant. In another embodiment of the present invention, the compositions may be substantially free of at least one other added nasal decongestant. In another embodiment of the present invention, the compositions may be substantially free of at least one other added opioid analgesic. In another embodiment of the present invention, the compositions may be substantially free of at least one other added expectorant. In other embodiments of the present invention, the compositions may be substantially free of one or more other added active ingredient, such as, but not limited to, antitussives, decongestants, nasal decongestants, opioid analgesics, and/or expectorants.

In other embodiments of the present invention, the compositions may additionally comprise one or more added active ingredients in addition to guaifenesin and hydrocodone bitartrate. For example, in one embodiment, the compositions of the present invention may comprise at least one other added antitussive. In another embodiment of the present invention, the compositions may comprise at least one added decongestant. In another embodiment of the present invention, the compositions may comprise at least one other added nasal decongestant. In another embodiment of the present invention, the compositions may comprise at least one other opioid analgesic. In another embodiment of the present invention, the compositions may comprise at least one other expectorant. In other embodiments of the present invention, the compositions may comprise one or more other active ingredient, such as, but not limited to, antitussives, decongestants, nasal decongestants, opioid analgesics, and/or expectorants.

Antitussives of interest include, but are not limited to, codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan hydrobromide, chlophendianol hydrochloride, carbetapentane citrate, caramiphen edisylate, noscapine, noscapine hydrochloride, and menthol, and functional variants and derivatives thereof.

Decongestants of interest include, but are not limited to, ephedrine, ephedrine sulfate, ephedrine hydrochloride, pseudoephedrine hydrochloride, phenylephrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride, and functional variants and derivatives thereof.

Opioid analgesics of interest include, but are not limited to, such as, codeine, morphine, hydromorphone, oxymorphone, levorphanol, fentanyl, propoxyphene, diphenoxylate, meperidine, methadone, oxycodone, butorphanol, benzonate and morphine.

Expectorants of interest include, but are not limited to ammonium chloride, ammonium carbonate, acetylcysteine, antimony potassium tartrate, glycerin, potassium iodide, sodium citrate, terpin hydrate, and tolu balsam.

### FORMULATIONS OF THE COMPOSITIONS OF THE INVENTION

A specific embodiment of the present invention may comprise swallowable compositions. Swallowable compositions are well known in the art and are those that do not readily dissolve when placed in the mouth and may be swallowed whole without any chewing or discomfort. In a specific embodiment of the present invention, the swallowable compositions may have a shape containing no sharp edges and a smooth and uniform surface. Among other dosage forms apparent to the skilled artisan, the solid oral dosage form may be a tablet, a discrete unit-filled capsule, or a sachet. In a specific embodiment, the dosage form is a caplet.

As used herein the term “tablet” refers to a medication, usually mixed with a binder powder, which is molded and pressed into the form of a tablet, traditionally circular or disk-shaped, but also oblong or differently shaped. As used herein, the term “capsule” refers to a solid dosage form in which the drug, in discrete units, is enclosed in a hard or soft soluble container, usually of a form of gelatin. The discrete units of the capsule dosage form include, but are not limited to, beads, granules, pellets, spheroids, particles, tablets, pills, etc. As used herein the term “sachet” refers to a packet which contains a powder containing the drug, which is to be dissolved in water and then taken orally. As used herein, the term “caplet” refers to a smooth, coated, oval-shaped tablet.

In some specific embodiments, the swallowable compositions may be formulated such that either guaifenesin and/or hydrocodone bitartrate are released in a controlled release manner into the blood stream after the composition is swallowed by the patient. In another embodiment, the swallowable compositions may be formulated such that guaifenesin is released in an immediate release manner after the composition is swallowed by the patient. In another embodiment, the swallowable compositions are formulated such guaifenesin released in a controlled manner into the blood stream after the composition is swallowed. In another specific embodiment, the swallowable compositions are formulated such that hydrocodone bitartrate released in a controlled manner into the blood stream after the composition is swallowed. In a specific embodiment, the controlled release may be an extended release. In another specific embodiment, the controlled release may be a delayed release. In another embodiment, the controlled release is a combination of delayed release and extended release. The controlled release of guaifenesin and/or hydrocodone may be achieved by the formulation of the dosage form, according to methods that are well known to those in the art.

To prepare the swallowable compositions in caplet form, each of the active ingredients may be combined in intimate admixture with a suitable carrier according to conventional compounding techniques. In a specific embodiment of swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia. Pharmaceutical carriers and formulations for swallowable compounds are well known to those of ordinary skill in the art. See generally, e.g., Wade & Waller, Handbook of Pharmaceutical Excipients (2nd ed. 1994).

Matrix. The term matrix, as used herein, refers to a solid material having an active agent incorporated therein. The skilled artisan should appreciate that the matrix material can be chosen from a wide variety of materials that can provide the desired dissolution profiles. Materials for the matrix can include, for example, one or more gel forming polymers such as polyvinyl alcohol, cellulose ethers including, for example, hydroxyl propyl alkyl, celluloses such as hydroxypropyl methyl cellulose, hydroxy alkyl celluloses such as hydroxy propyl cellulose, natural or synthetic gums such as guar gum, xanthum gum, and alginates, as well as, ethyl cellulose, polyethylene oxide, polyvinyl pyrrolidone, fats, waxes, polycarboxylic acids or esters such as the Carbopol® series of polymers, methacrylic acid copolymers, and methacrylate polymers. In addition to the above-mentioned ingredients, the matrix may also contain suitable quantities of other materials, for example, diluents, lubricants, binders, granulating aids, colorants, flavorants, and glidants that are conventional in the pharmaceutical arts. The quantities of these additional materials are sufficient to provide the desired effect to the desired formulation. A matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

The active ingredients of the present invention may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms. Pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols.

Disintegrants also may be included in the compositions of the present invention in order to facilitate dissolution. Disintegrants, including permeabilising and wicking agents, are capable of drawing water or saliva up into the compositions, which promotes dissolution from the inside as well as the outside of the compositions. Such disintegrants, permeabilising and/or wicking agents that may be used in the present invention include by way of example and without limitation, starches such as corn starch, potato starch, pre-gelatinized and modified starches thereof, cellulosic agents such as Ac-di-sol, montmorrilonite clays, cross-linked PVP, sweeteners, bentonite, microcrystalline cellulose, croscarmellose sodium, alginates, sodium starch glycolate, gums such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents such as hydroxymethylcellulose, hydroxypropylcellulose and hydroxyopropylmethylcellulose.

Finally, dissolution of the compositions may be facilitated by including relatively small particle sizes of the ingredients used.

In addition to those described above, any appropriate fillers and excipients may be utilized in preparing the swallowable compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium, and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, and polyols of less than 13 carbon atoms, such as mannitol, xylitol, sorbitol, maltitol, and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, and others known to those of ordinary skill in the art.

Glidants improve the flow of powder blends during manufacturing and minimize composition weight variation. Glidants that may be used in the present invention include by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents such as grape skin extract, beet red powder, beta carotene, annato, carmine, turmeric, paprika, and others known to those of ordinary skill in the art. It is recognized that no colorant is required in the nutritional supplement compositions described herein.

If desired, compositions may be sugar coated or enteric coated by standard techniques.

The swallowable compositions of the present invention may be prepared using conventional methods and materials known in the pharmaceutical art. For example, U.S. Pat. Nos. 5,215,754 and 4,374,082 relate to methods for preparing swallowable compositions. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in Wade & Waller, supra. All active ingredients, fillers and excipients are commercially available from companies such as Aldrich Chemical Co., FMC Corp, Bayer, BASF, Alexi Fres, Witco, Mallinckrodt, Rhodia, ISP, and others.

Controlled Release Formulations. While “immediate release” formulations release most of the drug into the blood stream almost immediately upon administration of the dosage form, “controlled release” formulations release the drug in a controlled release manner that can achieve various specific profiles of drug concentration in the blood plasma over time. The profile of the drug concentration in the blood stream over time depends not only in the release profile of the drug in the gastrointestinal tract of the patient, but also aspects such as the rate at which the active drug is absorbed into the blood stream, and rate at which the drug is cleared from the blood stream. A dosage form that results in the controlled release of the drug in the gastrointestinal tract of the patient may result in a long-term presence of the drug in the blood plasma of the patient. Therefore, a controlled release formulation may be used to decrease the number of times a patient needs to administer the dosage form per day, as well as provide a more uniform concentration of the drug in the blood plasma during the day.

There are several release profiles that can be created by current controlled release formulations by combinations of matrices, coatings and administration devices. An used herein, the term “controlled release” is used to mean any part of all of a dosage form that can release one or more active pharmaceutical agents continuously or intermittently over a prolonged period of time after administration (i.e., typically over a period of more than about 1 hour). The term “extended release,” as used herein, refers to the release of the drug in the gastrointestinal tract in a continuous and long-term manner that creates a long-term presence of the drug in the blood stream. The term “delayed release,” as used herein, refers to the release of discrete amount(s) of drug into the gastrointestinal tract some time after drug administration, e.g. enteric-coated products, and exhibits a lag time during which little or no absorption of the drug occurs. Delayed release is also termed “pulsatile release.”

Typically, controlled-release formulations provide an immediate release of drug which promptly produces the desired therapeutic effect, which then may be followed by a gradual and continual release of additional amounts of the drug to maintain this effect over a predetermined period of time. The lifespan of the drug in the bloodstream of the patient is one characteristic of the controlled release formulation. For example, a 12 h extended release formulation typically is one that results in a plasma drug concentration-time profile that has an extended concentration of the drug approaching but not beyond 12 hours. Further, the release techniques can be combined to further tailor the drug blood plasma concentration to fit the needs of the patient and the blood clearance properties of the drug. In various embodiments, substantially all of guaifenesin and/or hydrocodone bitartrate is released from the dosage form by about 4 hours, about 6 hours, about 8 hours, about 10 hours, about 12 hours, about 16 hours, about 20 hours and about 24 hours after ingestion by the patient.

Immediate release matrices. The immediate release matrices comprise the active agent combination and a disintegrant. Suitable disintegrants include, for example, starch, low-substitution hydroxypropyl cellulose, croscarmellose sodium, calcium carboxymethyl cellulose, hydroxypropyl starch, sodium starch glycolate, and microcrystalline cellulose. Upon exposure to a dissolution media, channels are formed in the solid material so that the active agent can escape. Dosage forms according to one embodiment of the present invention may be in the form of coated or uncoated matrices. Immediate release matrices may be formulated with extended release variants of drugs to create extended release formulations.

Immediate Release Coatings. A coating containing an immediate release drug can be added to the outside of the tablet cores to produce a final dosage form. Such a coating can be prepared by mixing the drug with polyvinylpyrrolidone (PVP) 29/32 or hydroxypropyl methylcellulose (HPMC) and water/isopropyl alcohol and triethyl acetate. Such an immediate release coating can be spray coated onto the tablet cores. The immediate release coating may also be applied using a press-coating process with a blend consisting of 80% by weight drug and 20% by weight of lactose and hydroxypropyl methylcellulose type 2910. Press coating techniques are known in the art and are provided in U.S. Pat. No. 6,372,254, incorporated herein by reference in its entirety.

In one embodiment, an immediate release matrix is formulated with an extended release variant of guaifenesin, such as potassium guaiacolsulfonate or guaifenesin tannate. In another embodiment, an immediate release matrix is formulated with extended release variant of hydrocodone bitartrate, such as hydrocodone polistirex or hydrocodone complexed with sodium polystyrene sulfonate (Amberlite™ IRP69). In another embodiment, an immediate release matrix is formulated with extended release variant of guaifenesin, such as potassium guaiacolsulfonate or guaifenesin tannate and extended release variant of hydrocodone bitartrate, such as hydrocodone polistirex. In other embodiments, the matrix is pressed into the dosage form and coated with an immediate release coating comprising guaifenesin.

Extended Release Formulations. The “extended release” or “sustained release” dosage form allows a reduction in dosing frequency to that presented by a conventional dosage form, e.g., a solution or immediate-release dosage form. In an extended release formulation, the matrix of the dosage form may comprise a combination of hydrophilic and hydrophobic polymers. In this embodiment, once administered, the hydrophilic polymer dissolves away to weaken the structure of the controlled release component and the hydrophobic polymer retards the water penetration and helps to maintain the shape of the drug delivery system.

As used herein, the term “polymer” includes single or multiple polymeric substances, which can optionally swell, gel, degrade or erode on contact with an aqueous environment (e.g., water). Examples include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelatinized starch, shellac, zein, and combinations thereof.

The term “hydrophilic polymers” as used herein includes one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone, of which hydroxypropyl methylcellulose is preferred. The term “hydrophilic polymers” can also include sodium carboxymethycellulose, hydroxymethyl cellulose, polyethelene oxide, hydroxyethyl methyl cellulose,. carboxypolymethylene, polyethelene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), an alkali metal or alkaline earth metal, carageenate alginates, ammonium alginate, sodium alganate, or mixtures thereof.

The “hydrophobic polymer” of the drug delivery system can be any hydrophobic polymer which retards the water penetration and helps to maintain the shape of the drug delivery system including, but not limited to, one or more polymers selected from carbomer, carnauba wax, ethylcellulose, glyceryl palmitostearate, shellac, zein, hydrogenated castor oil, hydrogenated vegetable oil type 1, microcrystalline wax, polacrilin potassium, polyethylene oxide, polymethacrylates, or stearic acid, of which hydrogenated vegetable oil type 1 is preferred. Hydrophobic polymers can include, for example, a pharmaceutically acceptable acrylic polymer, including, but not limited to, acrylic acid and methacrylic acid polymers and copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methyl methacrylate)copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. In alternate embodiments, the hydrophobic material is selected from materials such as one or more hydroxyalkyl celluloses such as hydroxypropyl methycellulose. The hydroxyalkyl cellulose is preferably a hydroxy (C₁ to C₆) alkyl cellulose, such as hydroxypropylcellulose, hydroxypropylmethylcellulose, or preferably hydroxyethylcellulose. The amount of the hydroxyalkyl cellulose in the present oral dosage form is determined, inter alia, by the precise rate of active agents desired and may vary from about 1% to about 80%. The polymers may also be pH dependent.

In one embodiment, an extended release matrix is formulated with guaifenesin. In another embodiment, an extended release matrix is formulated with hydrocodone bitartrate. In another embodiment, an extended release matrix is formulated with guaifenesin and hydrocodone bitartrate. In other embodiments, the matrix is pressed into the dosage form and coated with an immediate release coating or matrix comprising guaifenesin.

Delayed release formulations. Delayed release formulations are characterized by a lag followed by a rapid release of the drug. Various methods are available to generate the lag time, from coatings to various capsules, all of which will be well known to those in the art. In various embodiments of the compositions of the invention, the delayed release formulations can be combined with immediate release formulations, such as immediate release coatings, and extended release matrices. Further, delayed released formulations may be combined that have various release parameters, including varying lag times and additional drugs, to provide more complex profiles of drug blood levels.

In one embodiment, the delayed release system used is a capsular drug delivery system, which consists of a an insoluble capsule body housing a drug and a plug. The plug is removed after a predetermined lag time due to swelling, erosion or dissolution. One delayed system commercially available is the Pulsincap® system (Scherer DDS, Ltd.). For water insoluble drugs, rapid release can be achieved by the inclusion of effervescents agents or disintergrants. The plug material may consist of insoluble but permeable and swellable polymers (polymethacrylates, for example), erodible compressed polymers (hydroxypropylmethyl cellulose, polyvinyl alcohol, or polyethylene oxide, for example), congealed melted polymers (saturated polyglycolated glycerides glyceryl monooleate, for example), and enzymatically controlled erodible polymer (pectin, for example).

In another embodiment, the delayed release system used is a similar the commercially available system Port® System (Port Systems, LLC), which has a gelatin capsule coated with a semipermeable membrane housing and an insoluble plug, enclosing an osmotically active agent along with the drug formulation. When it capsule comes in contact with water, the water crosses the semipermeable membrane, the capsule swells, and the plug is ejected, releasing the drug. The lag time may be controlled by the coating thickness.

In another embodiment, the delayed release system used is a reservoir device with a delayed release coating. In this embodiment, the barrier coat erodes or dissolves after a specific lag period, and the drug is subsequently rapidly released. The lag period depends on the thickness of the coating. In this embodiment, the reservoir device may be any means by which the drug is held, including immediate release and extended release matrices. Commercially available systems using this methodology include the Time Clock® System (West Pharmaceutical Services Drug Delivery & Clinical Research Centre) (DE Pat. No. 4,122,039; Wilding et al., Int. J. Pharm. 111:99-102 (1994)) and Chronotropic® system (Gazzaniga et al., Int. J. Pharm. 2:77-83 (1994); Gassaniga et al., Eur. J. Biopharm. 40:246-250 (1994)). In another embodiment, a release pattern of two or more pulses can be obtained from a layered tablet such as provided in U.S. Pat. No. 4,865,849, which is expressly incorporated by reference herein. In other embodiments, rupturable coatings which depend on disintegration of the coating to release the drug, with the pressure necessary for the rupture of the coating achieved by effervescent excipients, swelling agents or osmotic pressure.

Delayed Release Coating._The delayed release profile of the formulations of the invention can be altered, for example, by increasing or decreasing the thickness of the delayed release coating, i.e., by varying the amount of overcoating. In some embodiments, the particles may be overcoated with an aqueous dispersion of a hydrophobic or hydrophilic material to modify the release profile. In some embodiments, the aqueous dispersion of hydrophobic material or film-former preferably further includes an effective amount of plasticizer, e.g., triethyl citrate. Preformulated aqueous dispersions of ethylcellulose, such as Aquacoat® (FMC Corp., Philadelphia Pa.), or Surelease® (Colorcon, West Point, Pa.), may be used. If Surelease® is used, it is not necessary to separately add a plasticizer. In some embodiments, the delay or lag time to the release of the drug can be predetermined by altering the coating thickness and/or composition of the coating, among others.

In embodiments of the present invention where the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the hydrophobic polymer, e.g., most often from about 1 percent to about 50 percent by weight of the hydrophobic polymer. Concentration of the plasticizer, however, is preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention. Examples of suitable plasticizers for the acrylic polymers of the present invention include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions (Rohm Pharma, Piscataway, N.J.) include polyethylene glycols, propylene glycol, diethyl phthalate, castor oil, and triacetin. It has further been found that addition of a small amount of talc reduces the tendency of the aqueous dispersion to stick during processing and acts a polishing agent.

One commercially available aqueous dispersion of ethylcellulose is Aquacoat® which is prepared by dissolving the ethylcellulose in a water-immiscible organic solvent and then emulsifying the ethylcellulose in water in the presence of a surfactant and a stabilizer. After homogenization to generate submicron droplets, the organic solvent is evaporated under vacuum to form a pseudolatex. The plasticizer is not incorporated into the pseudolatex during the manufacturing phase. Thus, prior to using the pseudolatex as a coating, the Aquacoat® is mixed with a suitable plasticizer.

Another aqueous dispersion of ethylcellulose is commercially available as Surelease®. This product is prepared by incorporating plasticizer into the dispersion during the manufacturing process. A hot melt of a polymer, plasticizer (dibutyl sebacate), and stabilizer (oleic acid) is prepared as a homogeneous mixture which is then diluted with an alkaline solution to obtain an aqueous dispersion which can be applied directly onto substrates.

In one embodiment, the acrylic coating is an acrylic resin lacquer used in the form of an aqueous dispersion, such as that which is commercially available from Rohm Pharma (Piscataway, N.J.) under the trade name Eudragit®. In additional embodiments, the acrylic coating comprises a mixture of two acrylic resin lacquers commercially available from Rohm Pharma under the trade names Eudragit® RL 30 D and Eudragit® RS 30 D. Eudragit® RL 30 D and Eudragit® RS 30 are copolymers of acrylic and methacrylic esters with a low content of quaternary ammonium groups, the molar ratio of ammonium groups to the remaining neutral (meth)acrylic esters being 1:20 in Eudragit® RL 30 and 1:40 in Eudragit® RS 30 D. The mean molecular weight is about 150,000 Daltons. The code designations RL (high permeability) and RS (low permeability) refer to the permeability properties of these agents. Eudragit® RL/RS mixtures are insoluble in water and in digestive fluids, however, coatings formed from them are swellable and permeable in aqueous solutions and digestive fluids.

The Eudragit® RL/RS dispersions may be mixed together in any desired ratio in order to ultimately obtain a controlled release formulation having a desirable dissolution profile. Desirable controlled release formulations may be obtained, for instance, from a delayed release coating derived from one of a variety of coating combinations, such as 100% Eudragit® RL; 50% Eudragit® RL and 50% Eudragit® RS; or 10% Eudragit® RL and Eudragit® 90% RS. One skilled in the art should recognize that other acrylic polymers may also be used, for example, Eudragit® L. In addition to modifying the dissolution profile by altering the relative amounts of different acrylic resin lacquers, the dissolution profile of the ultimate product may also be modified, for example, by increasing or decreasing the thickness of the delayed release coating.

Enteric coating. In one embodiment, the delayed release coating is an enteric coating. All commercially available pH-sensitive polymers may be used to form the enteric coating. The drug coated with the enteric coating is minimally or not released in the acidic stomach environment of approximately below pH 4.5, but not limited to this value. The drug should become available when the enteric layer dissolves at the higher pH; after a suitable delayed time; or after the unit passes through the stomach. A specific duration of drug release time is in the range of up to 7 hours after dosing under fasting conditions.

Enteric polymers include cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters such as, for instance, materials known under the trade name Eudragit® L12.5, Eudragit® L100, or Eudragit® S12.5, S100 (Rohm Pharma, Piscataway N.J.) or similar compounds used to obtain enteric coatings. Aqueous colloidal polymer dispersions or re-dispersions can be also applied, e.g., Eudragit® L30 D-55, Eudragit® L100-55, Eudragit® S100, Eudragit® preparation 4110Dc; Aquateric®, Aquacoat® CPD 30 (FMC Corp.); Kollicoat MAE® 30D and Kollicoat MAE® 30DP (BASF); Eastacryl® 30D (Eastman Chemical, Kingsport, Tenn.).

The enteric polymers can be modified by mixing with other known coating products that are not pH sensitive. Examples of such coating products include the neutral methacrylic acid esters with a small portion of trimethylammonioethyl methacrylate chloride, sold currently under the trade names E Eudragit®, Eudragit® RL, Eudragit® RS; a neutral ester dispersion without any functional groups, sold under the trade names Eudragit® NE30D and Eudragit® NE30; and other pH independent coating products.

The release of the therapeutically active agent from the delayed release formulation of the present invention can be further influenced, i.e., adjusted to a desired rate, by the addition of one or more release-modifying agents. The release-modifying agent may be organic or inorganic and include materials that can be dissolved, extracted, or leached from the coating in the environment of use. The pore-formers may comprise one or more hydrophilic materials such as hydroxypropyl methylcellulose. The release-modifying agent may also comprise a semi-permeable polymer. In certain embodiments, the release-modifying agent is selected from hydroxypropyl methylcellulose, lactose, metal stearates, and mixtures thereof.

Any the of the coating solutions disclosed herein may contain, in addition to the film-former, plasticizer, and solvent system (i.e., water), a colorant to provide elegance and product distinction, as well as protect light sensitive drugs in the underlying layers. Color may be added to the solution of the therapeutically active agent instead of, or in addition to the aqueous dispersion of hydrophobic material. For example, color may be added to Aquacoat® via the use of alcohol or propylene glycol based color dispersions, milled aluminum lakes and opacifiers such as titanium dioxide by adding color with shear to the water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retardant effect of the coating.

In one embodiment, an immediate release matrix is formulated with guaifenesin, and the dosage form is coated with an extended release coating. In another embodiment, an immediate release matrix is formulated with hydrocodone bitartrate, and the dosage form is coated with an extended release coating. In another embodiment, an immediate release matrix is formulated with guaifenesin and hydrocodone bitartrate, and the dosage form is coated with an extended release coating. In other embodiments, the extended release coating is further coated with an immediate release coating comprising guaifenesin.

Particulate dosage forms. In some embodiments, a delayed release formulation is made that mimics a controlled release effect by combining several delayed release formulations with different lag times into one dosage form. For example, solid controlled release particles with varying lag times may be placed in a gelatin capsule in an amount sufficient to provide an effective controlled release dose when ingested and contacted by an environmental fluid, e.g., gastric fluid, intestinal fluid or dissolution media. The term “particle” as used herein means a granule having a diameter of between about 0.01 mm and about 5.0 mm, specifically between about 0.1 mm and about 2.5 mm, and more specifically between about 0.5 mm and about 2 mm. The skilled artisan should appreciate that particles according to the present invention can be any geometrical shape within this size range and so long as the mean for a statistical distribution of particles falls within the particle sizes enumerated above, they will be considered to fall within the contemplated scope of the present invention. Particles can assume any standard structure known in the pharmaceutical arts. Such structures include, for example, matrix particles, non-pareil cores having a drug layer and active or inactive cores having multiple layers thereon. The particles can be made by mixing the relevant ingredients and granulating the mixture. The resulting particles are dried and screened, and the particles having the desired size are used for drug formulation. A controlled release coating can be added to any of these structures to create a controlled release particle.

In one embodiment, oral dosage forms are prepared to include an effective amount of particles as described above within a capsule. For example, melt-extruded particles may be placed in a gelatin capsule in an amount sufficient to provide an effective controlled release dose when ingested and contacted by gastric fluid. In another embodiment, a suitable amount of the particles are compressed into an oral tablet using conventional tableting equipment using standard techniques. Techniques and compositions for making tablets (compressed and molded), capsules (hard and soft gelatin), and pills are also described in Remington's Pharmaceutical Sciences, Arthur Osol, ed., 1553-93 (1980), incorporated herein by reference.

In one embodiment, particles are formulated from an immediate release matrix and guaifenesin, and portions of the particles are coated with delayed release coating of various lag profiles, and put into a gelatin capsule. In another embodiment, particles are formulated from an immediate release matrix and hydrocodone bitartrate, and portions of the particles are coated with delayed release coating of various lag profiles, and put in a gelatin capsule. In another embodiment, particles are formulated from an immediate release matrix and guaifenesin and hydrocodone bitartrate, and portions of the particles are coated with delayed release coating of various lag profiles, and put in a gelatin capsule. In other embodiments, the particles coated with an immediate release coating comprising guaifenesin.

Manufacture of Formulations. In addition, the formulation of respective release components can occur by appropriate granulation methods as is well known in the art. In wet granulation, solutions of the binding agent (polymer) are added with stirring to the mixed powders. The powder mass is wetted with the binding solution until the mass has the consistency of damp snow or brown sugar. The wet granulated material is forced through a sieving device. Moist material from the milling step is dried by placing it in a temperature controlled container. After drying, the granulated material is reduced in particle size by passing it through a sieving device. Lubricant is added, and the final blend is then compressed into a matrix dosage form.

In fluid-bed granulation, particles of inert material and/or active agent are suspended in a vertical column with a rising air stream. While the particles are suspended, a common granulating material in solution is sprayed into the column. There is a gradual particle buildup under a controlled set of conditions resulting in tablet granulation. Following drying and the addition of lubricant, the granulated material is ready for compression.

In dry-granulation, the active agent, binder, diluent, and lubricant are blended and compressed into tablets. The compressed large tablets are comminuted through the desirable mesh screen by sieving equipment. Additional lubricant is added to the granulated material and blended gently. The material is then compressed into tablets. Such tablet cores can be used for further processing as bilayer tablets, press coated tablets, or film coated tablets.

Application of coatings. One or more coatings as described herein may be added to the surface of any spheroid or bead, a tablet or caplet, or any other solid dosage form by spraying the solution onto the dosage form, for example, non pareil 18/20 beads, using a Wuster insert. Optionally, additional ingredients are also added prior to coating the beads in order to assist the binding of the active agents to the beads, and/or to color the solution, etc. For example, a product that includes hydroxypropyl methylcellulose with or without colorant (e.g., Opadry®, commercially available from Colorcon, Inc.) may be added to the solution and the solution mixed (e.g., for about 1 hour) prior to application onto the beads. The resultant coated substrate, beads in this example, may then be optionally overcoated with a barrier agent to separate a therapeutically active agent coating from a hydrophobic controlled release coating, for example. An example of a suitable barrier agent is one that comprises hydroxypropylmethylcellulose. However, any film-former known in the art may be used. In one embodiment, the barrier agent does not affect the dissolution rate of the final product.

In embodiments of the present invention, the stabilized product is obtained by subjecting the coated substrate to oven curing at a temperature above the T_{g} of the plasticized acrylic polymer for the required time period, the optimum values for temperature and time for the particular formulation being determined experimentally. In certain embodiments of the present invention, the stabilized product is obtained via an oven curing conducted at a temperature of about 45° C. for a time period from about 1 to about 48 hours. It is also contemplated that certain products coated with controlled release coatings require a curing time longer than 24 to 48 hours, e.g., from about 48 to about 60 hours or more.

Packaging. A specific embodiment of the present invention may comprise swallowable compositions of dosage forms packaged in blister packs. Blister packs as packaging for swallowable compositions are well known to those of ordinary skill in the art. Blister packs may be made of a transparent plastic sheet which as been formed to carry a matrix of depression or blisters. One or more swallowable compositions are received in each depression or blister. A foil or plastic backing is then adhered across the plane of the sheet sealing the swallowable compositions in their respective blisters. Examples of materials used for the blister packs include, but are not limited to, aluminum, paper, polyester, PVC, and polypropylene. Alternative materials are known to those of ordinary skill in the art. To remove a swallowable composition, the depression material is pressed in and the composition is pushed through the backing material. Multiple blister packs may be placed in an outer package, often a box or carton, for sale and distribution.

Another specific embodiment of the present invention may comprise swallowable compositions packaged in bottles. The bottle may be glass or plastic in form with a pop or screw top cap. Bottle packaging for compositions in swallowable form are well known to those of ordinary skill in the art.

Additionally, the dosage forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

Kits are commonly used for dispensing pharmaceutical actives and are known in the pharmaceutical art. Kits were developed and designed to administer multiple doses of the same active ingredient or for the concurrent or nonconcurrent administration of two or more active agents. See e.g. U.S. Pat. No. 6,024,222, to Friberg et al., issued Feb. 15, 2000; U.S. Pat. No. 6,219,997, to Friberg et al., issued Apr. 24, 2001; U.S. Pat. Pub. 2003/0168376 A1, Taneja et al. published Sep. 11, 2003; U.S. Pat. Pub. 2003/0111479, Taneja et al., published Jun. 19, 2003; U.S. Pat. No. 6,375,956, to Hermelin et al., issued Apr. 23, 2002; PCT Pub. WO 88/02342, Astra Lakemedel Aktiebolag, published Apr. 7, 1988; U.S. Pat. No. 4,295,567, to Knudsen, issued Oct. 20, 1981; DE 29719 070, to Byk Gulden Lomberg Chemische Fabrik, published Jun. 25, 1998; U.S. Pat. No. 5,848,976, to Weinstein, issued Dec. 15, 1998; U.S. Pat. No. 6,270,796, to Weinstein, issued Aug. 7, 2001; U.S. Pat. No. 6,564,945, to Weinstein et al., issued May 20, 2003; and U.S. Pat. No. 5,788,974, to D'Amico et al., issued Aug. 4, 1998.

A specific embodiment of the present invention may comprise kits wherein the packaging may consist of bottles and blister packs. In another embodiment, the kits may contain bottles that are sold together, one bottle containing a first composition and a second bottle containing a second composition. In a further embodiment, the kits may contain bottles that are sold separately; one bottle containing a first composition and a second bottle containing a second composition. In yet another embodiment, the kits may contain bottles containing a first composition and a second composition that are advertised as more effective if co-administered. In another embodiment, the advertisements of the kits may consist of internet, print, and product packaging advertisements. In a further embodiment, the kits may contain blister packs that are sold together wherein the blister packs may contain a first blister pack containing a first composition and a second blister pack containing a second composition. In another embodiment, the kits may contain blister packs that contain both a first composition and a second composition paired together per unit dose. In yet another embodiment, the kits may contain blister packs that are sold separately that comprise a first blister pack containing a first composition and a second blister pack containing a second composition. In another embodiment, the kits may contain blister packs containing a first composition and a second composition that are advertised as more effective if co-administered. In a further embodiment, the kits may contain a first composition and a second composition that may be co-administered to a patient. In another embodiment, the kits may contain a first composition and a second composition that may be co-administered to a patient orally.

Methods to treat patients. Methods to treat patients with the compositions of the present invention may be specifically administered in amounts to patients that alleviate symptoms, such as coughing, sneezing, rhinorrhea, and/or nasal obstruction, caused by a variety of conditions. An exemplary dosage of the compositions of the present invention may consist of one or more caplets for human oral consumption. If more than one caplet is used, each individual caplet may be identical to the other caplets, or each may contain only some of the ingredients of the composition, so that the combination of the different caplets comprises a composition of the present invention.

The compositions and methods of the present invention may alleviate symptoms, such as coughing, sneezing, rhinorrhea, and/or nasal obstruction, caused by a variety of conditions. For instance, coughing, sneezing, rhinorrhea, and/or nasal obstruction may be caused by, for example and without limitation, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, or a respiratory disease, such as, for example and without limitation, a cold, acute bronchitis, chronic bronchitis, asthmatic bronchitis, bronchiectasis, pneumonia, lung tuberculosis, silicosis, silicotuberculosis, pulmonary cancer, upper respiratory inflammation (caused by, for example and without limitation, pharyngitis, laryngitis, nasal catarrh), asthma, bronchial asthma, infantile asthma, pulmonary emphysema, pneumoconiosis, pulmonary fibrosis, pulmonary silicosis, pulmonary suppuration, pleuritis, tonsillitis, cough hives, or whooping cough. The compositions and methods of the present invention may provide relief from symptoms caused by all of the above.

Another aspect of the invention is a method of administering to a patient the composition of the invention, comprising immediate release guaifenesin, extended release guaifenesin and extended release hydrocodone bitartrate. In some embodiments, the composition is administered to the patient orally. The composition of the invention may be administered in varying volumes and at varying frequencies. In specific embodiments, the dose volume is from 0.5 to 3.0 dosage forms. Specific dosages include, but are not limited to, 0.5, 1.0, 2.0 or 3.0 dosage forms. The frequency of the dose may vary from every other day to several times a day. In specific embodiments, the frequency of administration may be once a day, twice a day, three times a day or four times a day. In other specific embodiments, the frequency of the dose may be once a day or twice a day.

Total daily dosages of the compounds useful according to this invention administered to a host in single or divided doses are generally in amounts of from about 0.01 mg/kg to about 100 mg/kg body weight daily, and preferably from about 0.05 mg/kg to about 50 mg/kg body weight daily. It should be understood, however, that the specific dose level for any particular patient will depend upon a variety of factors including body weight, general health, gender, diet, time and route of administration, rates of absorption and excretion, combination with other drugs, and the severity of the particular disease being treated. Actual dosage levels of active ingredient in the compositions of the present invention may be varied so as to obtain an amount of active ingredient that is effective to obtain a desired therapeutic response for a particular composition and method of administration. The selected dosage level, therefore, depends upon the desired therapeutic effect, on the route of administration, on the desired duration of treatment, and other factors. Total daily dose of the compounds useful according to this invention administered to a host in single or divided doses may be in amounts, for example, of from about 0.01 mg/kg to about 20 mg/kg body weight daily and preferably 0.02 to 10 mg/kg/day.

The compounds useful according to this invention may be administered to an adult patient as a dose, to be taken once every 12 hours. It should be understood, however, that the specific dose level for any particular patient may depend upon a variety of factors including body weight, general health, gender, diet, time and route of administration, rates of absorption and excretion, combination with other drugs, and the severity of the particular disease being treated. For example, adults and children 12 years of age and older may be administered ½ to one dose every 12 hours. Children may be from 6 to 12 years of age may be administered ½ dose every 12 hours.

In a specific embodiment of the compositions and methods of the present invention, an immediate release form of guaifenesin may be included in amounts ranging from about 190 mg per dose to about 210 mg per dose. In another specific embodiment of the compositions and methods of the present invention, an immediate release form of guaifenesin may be included in amounts ranging from about 180 mg per dose to about 220 mg per dose. In another specific embodiment of the compositions and methods of the present invention, an immediate release form of guaifenesin may be included in amounts ranging from about 150 mg per dose to about 250 mg per dose. In another specific embodiment of the compositions and methods of the present invention, an immediate release form of guaifenesin may be included in an amount of about 200 mg per dose.

In one specific embodiment, the dosage range of guaifenesin per 12 hours is from about 570 mg to about 630 mg. In another specific embodiment, the dosage range of guaifenesin per 12 hours is from about 540 mg to about 660 mg. In another specific embodiment, the dosage range of guaifenesin per 12 hours is from about 500 mg to about 700 mg. In another specific embodiment, the dosage of guaifenesin per 12 hours is about 600 mg. In other embodiments, the guaifenesin in any of these embodiments is substituted by a dose equivalent amount of variant or derivative of guaifenesin with similar therapeutic activity. Dosage forms according to the present invention may contain such amounts or fractions thereof as may be used to make up the daily dose.

In one specific embodiment, the dosage range of hydrocodone bitartrate per 12 hours is from about 5.2 mg to about 5.8 mg. In another specific embodiment, the dosage range of hydrocodone bitartrate per 12 hours is from about 5.0 mg to about 6.1 mg. In another specific embodiment, the dosage range of hydrocodone bitartrate per 12 hours is from about 4.1 mg to about 6.4 mg. In another specific embodiment, the dosage of hydrocodone bitartrate per 12 hours is about 5.5 mg. In other embodiments, the hydrocodone bitartrate in any of these embodiments is substituted by a dose equivalent amount of variant or derivative of hydrocodone bitartrate with similar therapeutic activity. Dosage forms according to the present invention may contain such amounts or fractions thereof as may be used to make up the daily dose.

In some embodiments, the patient is a human over about 12 years of age and the composition may be administered in about 1 to 2 dosage forms, once or twice a day. In other embodiments, the patient is human from about 6 to about 12 years of age, and the composition of the invention is administered in about ½ dosage form once a day or twice a day. In another embodiment, the patient is a human from about 2 to about 6 years of age, and the composition of the invention is administered in an about 1.25 ml to about 2.5 ml dose once a day or twice a day.

The total dosage per day of the active compounds may be a factor in determining the criteria for administering the composition of the invention. For example, compositions with a higher concentration of active compounds may be taken in smaller dosages and/or less frequently, and compositions with lower concentrations of the active compounds may be taken in larger volume dosages and/or more frequently.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation is prepared in swallowable form containing the following active ingredients per 2 caplets:

| | |
|---|---|
| Immediate release guaifenesin | 200 mg |
| Extended release guaifenesin | 400 mg |
| Extended release hydrocodone bitartrate | 5.5 mg |

### Example 2

A study is undertaken to evaluate the effectiveness of the compositions of the present invention in the treatment of patients. The objective of the study is to determine whether oral intake of the compositions of the present invention results in an improvement of the symptoms of coughing, sneezing, rhinorrhea, and/or nasal obstruction.

A double-blind, placebo controlled study is conducted over a three-day period. A total of 120 subjects, all presenting for treatment of symptoms of coughing, sneezing, rhinorrhea, and/or nasal obstruction, are chosen for the study. The patients range in age from 12 to 72 years old.

An initial assessment of the symptoms of each patient is conducted when the patients initially present for treatment. The treating physician rates the severity of the symptoms on a 4-point scale (0: absent; 1: mild; 2: moderate; 3: severe). For inclusion in the study, a patient must be rated with a score of two or above for cough and a total score of at least 5 for the sum of the four selected symptoms.

The 120 subjects chosen for the study are separated into two separate groups of 60. The characteristics of the symptoms between the two groups are comparable. The first group is administered a 2 caplet dose of the composition of the present invention every twelve hours for three days. The second group is administered a placebo medication every twelve hours for three days that is similar in all respects to the administered composition except for the exclusion of the active ingredients, hydrocodone bitartrate and guaifenesin. No other medications are taken by the patients during the assessment period.

Patients self-evaluate their symptoms of coughing, sneezing, rhinorrhea, and nasal obstruction using the same 4-point scale (0: absent; 1: mild; 2: moderate; 3: severe) thirty minutes after each dose administration. Patients also note the presence and severity of adverse effects of taking the medication on the 4-point scale. In addition to the initial assessment on day 1, patients are evaluated at the end of day two and day three by the treating physician.

The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at each dose administration, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

This study will demonstrate the efficacy of the composition of the present invention in treating the symptoms of coughing, sneezing, rhinorrhea and nasal obstruction. Regarding potential adverse effects of taking the medication, if there are no significant differences between the two therapeutic groups, this study will demonstrate that the administration of the composition of the present invention is effective at treating symptoms of coughing, sneezing, rhinorrhea, and/or nasal obstruction, in addition to being well-tolerated by the patients.

While specific embodiments of the present invention have been described, other and further modifications and changes may be made without departing from the spirit of the invention. All further and other modifications and changes are included that come within the scope of the invention as set forth in the claims. The disclosures of all publications cited above are expressly incorporated by reference in their entireties to the same extent as if each were incorporated by reference individually.

What is claimed is:

1. A composition comprising hydrocodone bitartrate and guaifenesin, wherein said composition is administrable to a patient.

2. The composition of claim 1, wherein said composition is substantially free of other added active ingredients.

3. The composition of claim 2, wherein said other added active ingredient is another antitussive.

4. The composition of claim 3, wherein said another antitussive comprises one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

5. The composition of claim 2, wherein said other added active ingredient is a decongestant.

6. The composition of claim 5, wherein said decongestant comprises a nasal decongestant.

7. The composition of claim 6, wherein said nasal decongestant comprises one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, phenylephrine and xylometazoline hydrochloride.

8. The composition of claim 2, wherein said other added active ingredient is another opioid analgesic.

9. The composition of claim 8, wherein said another opioid analgesic comprises one or more of the group consisting of codeine, morphine, hydromorphone, oxymorphone, levorphanol, fentanyl, propoxyphene, diphenoxylate, meperidine, methadone, and oxycodone.

10. The composition of claim 2, wherein said other added active ingredient is another expectorant.

11. The composition of claim 10, wherein said another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

12. The composition of claim 1, wherein said composition is in a dosage form that is solid.

13. The composition of claim 12, wherein dosage form is a caplet.

14. The composition of claim 1, wherein the guaifenesin comprises immediate release and controlled release forms.

15. The composition of claim 14, wherein the controlled release form of guaifenesin is at least one selected from the group consisting of guaiacolsulfonate and guaifenesin tannate.

16. The composition of claim 14, wherein controlled release and immediate release forms of guaifenesin are formulated in an immediate release matrix.

17. The composition of claim 14, wherein the controlled release form of guaifenesin is formulated in an immediate release matrix and the dosage form is coated with an immediate release coating comprising guaifenesin.

18. The composition of claim 1, wherein the hydrocodone bitartrate comprises a controlled release form.

19. The composition of claim 18, wherein the controlled release form is at least one selected from the group consisting of hydrocodone polistirex and a resinate of hydrocodone.

20. The composition of claim 19, wherein the resinate of hydrocodone is hydrocodone complexed with sodium polystyrene sulfonate.

21. The composition of claim 18, wherein the controlled release form of hydrocodone bitartrate is formulated in an immediate release matrix.

22. The composition of claim 12, wherein the dosage form is formulated so that hydrocodone bitartrate is released in a controlled release manner and guaifenesin is released in an immediate release and a controlled release manner.

23. The composition of claim 22, wherein substantially all of the drugs are released from the dosage form by about 12 hours after ingestion by a patient.

24. The composition of claim 22, wherein the hydrocodone bitartrate is formulated in an immediate release matrix and the resulting dosage form is coated with a delayed release coating.

25. The composition of claim 24, wherein the dosage form is additionally coated with an exterior coating of guaifenesin in an immediate release coating.

26. The composition of claim 22, wherein part or all of the guaifenesin is formulated in an immediate release matrix and the resulting dosage form is coated with a delayed release coating.

27. The composition of claim 26, wherein the dosage form is additionally coated with an exterior coating of guaifenesin in an immediate release coating.

28. The composition of claim 24 which comprises a particle comprising hydrocodone bitartrate in an immediate release matrix with a delayed release coating.

29. The composition of claim 26 which comprises a particle comprising guaifenesin in an immediate release matrix with a delayed release coating.

30. A composition, in which the particles of claims 28 are combined with particles comprising guaifenesin in an immediate release matrix with a delayed release coating; and are enclosed in a capsule or compressed into a tablet.

31. The composition of claim 30, wherein the delayed release coating is of varied compositions and thicknesses.

32. The composition of claim 30, further comprising particles of guaifenesin in an immediate release matrix without a delay release coating.

33. The composition of claim 30, further comprising particles having an exterior coating of guaifenesin in an immediate release coating.

34. The composition of claim 22, wherein at least part of the guaifenesin is formulated in an extended release matrix.

35. The composition of claim 34, wherein the dosage form is coated with guaifenesin in a immediate release matrix or coating.

36. The composition of claim 22, wherein the hydrocodone bitartrate is formulated in an extended release matrix.

37. The composition of claim 1, wherein said composition comprises about 2.7 mg per dose to about 8.3 mg per dose controlled release hydrocodone bitartrate.

38. The composition of claim 1, wherein said composition comprises about 200 mg per dose to about 600 mg per dose controlled release guaifenesin.

39. The composition of claim 1, wherein said composition comprises about 100 mg per dose to about 300 mg per dose immediate release guaifenesin.

40. The composition of claim 1, wherein said composition comprises about 4.1 mg per dose to about 6.9 mg per dose controlled release hydrocodone bitartrate.

41. The composition of claim 1, wherein said composition comprises about 350 mg per dose to about 450 mg per dose controlled release guaifenesin.

42. The composition of claim 1, wherein said composition comprises about 150 mg per dose to about 250 mg per dose immediate release guaifenesin.

43. The composition of claim 1, wherein said composition comprises about 5.0 mg per dose to about 6.1 mg per dose controlled release hydrocodone bitartrate.

44. The composition of claim 1, wherein said composition comprises about 360 mg per dose to about 440 mg per dose controlled release guaifenesin.

45. The composition of claim 1, wherein said composition comprises about 180 mg per dose to about 220 mg per dose immediate release guaifenesin.

46. The composition of claim 1, wherein said composition comprises about 5.2 mg per dose to about 5.8 mg per dose controlled release hydrocodone bitartrate.

47. The composition of claim 1, wherein said composition comprises about 380 mg per dose to about 420 mg per dose controlled release guaifenesin.

48. The composition of claim 1, wherein said composition comprises about 190 mg per dose to about 210 mg per dose immediate release guaifenesin.

49. The composition of claim 1, wherein said composition comprises about 5.2 mg per dose to about 5.8 mg per dose controlled release hydrocodone bitartrate; about 380 mg per dose to about 420 mg per dose controlled release guaifenesin; and about 190 mg per dose to about 210 mg per dose immediate release guaifenesin.

50. The composition of claim 1, wherein said composition comprises about 5.5 mg per dose controlled release hydrocodone bitartrate.

51. The composition of claim 1, wherein said composition comprises about 400 mg per dose controlled release guaifenesin.

52. The composition of claim 1, wherein said composition comprises about 200 mg per dose immediate release guaifenesin.

53. The composition of claim 1, wherein said composition comprises about 5.5 mg per dose controlled release hydrocodone bitartrate; about 400 mg per dose controlled release guaifenesin; and about 200 mg per dose immediate release guaifenesin.

54. A method comprising administering to a patient a composition comprising the composition of claim 1.

55. The method of claim 54, wherein the composition is administered to the patient orally.

56. The method of claim 54 wherein the composition is administered to the patient at a frequency selected from the group consisting of once a day, twice a day, three times a day and four times a day.

57. The method of claim 54, wherein the composition is administered to the patient in a dose selected from the group consisting of 0.5, 1, 1.5, 2, 2.5 and 3 dosage forms.

58. The method of claim 54, wherein the patient is suffering from at least one condition selected from the group consisting of coughing, sneezing, rhinorrhea, nasal obstruction, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, a respiratory disease, a cold, acute bronchitis, chronic bronchitis, asthmatic bronchitis, bronchiectasis, pneumonia, lung tuberculosis, silicosis, silicotuberculosis, pulmonary cancer, upper respiratory inflammation, pharyngitis, laryngitis, nasal catarrh, asthma, bronchial asthma, infantile asthma, pulmonary emphysema, pneumoconiosis, pulmonary fibrosis, pulmonary silicosis, pulmonary suppuration, pleuritis, tonsillitis, cough hives, and whooping cough.
